(19) United States
(12) Patent Application Publication
(10) Pub. No.: US 2004/0220675 A1
(43) Pub. Date: Nov. 4, 2004

(54) Total elbow replacement for dogs

(76) Inventors: Ralph Harrison Lewis, Lakeport, CA (US); Toni Lewis, Lakeport, CA (US)

Correspondence Address: Bruce H. Johnsonbaugh, Eckhoff & Hoppe, 333 Sacramento Street, San Francisco, CA, 94111, US

(21) Appl. No.: 10/832,787

(22) Filed: Apr. 26, 2004

Related U.S. Application Data
(60) Provisional application No. 60/466,763, filed on Apr. 30, 2003.

(51) Int. Cl. A61F 2/38

(52) U.S. Cl. 623/20.11; 623/23.39

(57) ABSTRACT

A total elbow replacement prosthesis for dogs is provided. A humeral component has an elongated stem carried by the humerus and a generally cylindrical head carried by the stem. The cylindrical head has a spiral groove formed at an angle substantially the same as the angle of the trochlear notch in a healthy ulna. A radial ulnar component is provided having a body and a formed plastic seat against which the head of the humeral component sits and articulates. The plastic seat includes a vertical ridge which engages the spiral groove to provide lateral support for the prosthesis and to mimic the natural motion of a healthy elbow. The radial ulnar component is anchored to the ulnar. A radial cap is carried by the proximal end of the radius and has a domed surface adapted to engage a recess in the body of the radial ulna component. The prosthesis is therefore unconstrained and the radius and ulna are free to rotate relative to each other about their longitudinal axes to allow natural pronation and supination of the carpus.

### CROSS REFERENCE TO RELATED APPLICATION

This application claims the benefit of and priority from U.S. provisional application Serial No. 60/466,763 filed on Apr. 30, 2003.

### BACKGROUND AND BRIEF SUMMARY OF INVENTION

The present invention provides a total elbow replacement for dogs. More particularly, the present invention provides for the first time an unconstrained full elbow implant for dogs which allows motion of the joint with essentially the same degree of freedom and support as in a healthy natural joint.

The need for an effective canine total elbow implant is clear. Numerous articles have been written on the subject and at least two U.S. patents document the need. Those patents are Conzemius U.S. Pat. Nos. 6,162,253 and 6,306,171. Although the Conzemius patents provide total replacement elbow joints, each has potential deficiencies as used in dogs.

The Conzemius '253 patent provides a radioulnar component having stems 21 and 22 which are embedded in and cemented to the radius and ulna, respectively. When cemented in place, the stems 21 and 22 prevent relative rotation of the radius and ulna about their longitudinal axes, the normal range of which in dogs is approximately 30 degrees. By preventing relative rotation of the radius and ulna, the device of the Conzemius '253 patent may result in either pain experienced by the dog in attempting to pronate or supinate the carpus, or loosening of the stems 21 and 22 embedded in the radius and ulna, or both. The present invention allows relative rotation of the radius and ulna, thereby allowing pain-free pronation and supination of the carpus of the dog and without any tendency to loosen the implant from its anchorage in the joint.

The prior art has clearly documented the normal range of rotational, or supinational, movement of the canine elbow and carpal joints. In Miller's “Anatomy of the Dog,” Second Edition, at p. 243, the authors note that the normal range of supination of the canine forepaw (which includes rotation of the elbow and carpus) is approximately 90 degrees. The normal rotation of the elbow joint is simply not allowed by the prosthesis shown in the Conzemius '253 patent.

Another significant drawback of the Conzemius '253 patent is that extension of the joint in weight-bearing differs significantly from the extension in a healthy, natural elbow. The present invention provides a closer motion to the natural motion by providing a trochlear groove which forms a spiral around the distal humerus. The spiral groove provides a more natural motion as the joint extends in weight bearing, since the humerus moves medially in relation to the radial and ulnar joint surfaces. By designing the prosthesis to more closely mimic the motion of a healthy natural joint, loosening of the prosthesis, as well as pain experienced by the dog, are reduced.

The prior art has recognized the inclination of the proximal ulna in normal canine elbow joints. In “Manual of Small Animal Arthrology” by Houlton and Collinson, 1994, p. 176, the inclination is described and illustrated. The present invention includes a spiral groove around the distal humerus to mimic the natural motion. This natural motion is not allowed in prior art canine elbow prostheses.

The Conzemius '171 patent provides a ball and socket joint having only a single anchoring stem carried by the radius. Although this device allows relative rotation between the radius and ulna, it has drawbacks as used in dogs. The primary drawback is that the ball and socket design does not provide side-to-side or lateral support as the joint articulates about a horizontal axis. The Conzemius '171 patent proposes the use of the bell and socket device in humans as well as in canines. The use of the ball in humans with its associated lack of side-to-side or lateral support is not as serious because the human joint is not a weight bearing joint. However, in the case of dogs and other quadrupeds, the lack of side-to-side or lateral support becomes more serious. The natural healthy canine elbow provides significant side-to-side or lateral support by the interaction of the trochlear notch of the ulna relative to the grooved, distal end of the humerus.

The prior art has clearly recognized the lateral support in a natural elbow joint. At page 243 of “Anatomy of the Dog,” supra, the author notes:

“***Lateral movements of the elbow joint are minimal because of the strong collateral ligaments and the forward protrusion of the anconeal process of the ulna into the deep olecranon fossa of the humerus.***”

The present invention overcomes each of the above-noted deficiencies of the elbow replacements shown and described in both Conzemius patents.

A primary object of the invention is to provide a total elbow replacement for dogs which is unconstrained and which provides side-to-side or lateral support for the joint.

A further object of the invention is to provide a total elbow replacement for dogs which allows pronation and supination of the forepaw of the dog, thereby reducing the tendency of the implant to loosen from its anchorage and also reducing pain experienced by the dog.

Other objects and advantages will become apparent from the following description and drawings wherein:

### BRIEF DESCRIPTION OF THE DRAWINGS

FIG. 1 is a perspective view of the humeral component of the invention;

FIG. 2 is a perspective view of the radial ulnar component of the invention;

FIG. 3 is a perspective view showing the implant in place and illustrating in phantom the humerus, radius and ulna bones of the dog;

FIG. 4 is a schematic representation showing how the concavity or recess formed in the body of the radial ulnar component interacts with the domed head of the radial cap;

FIG. 5 is a schematic representation illustrating how the spiral groove in the humeral component is inclined relative to longitudinal axis X-X of the humeral component;

FIG. 6 is a perspective and cranial view of a natural, healthy left ulna, illustrating the inclination of the trochlear notch;

FIG. 7A is a schematic representation of the invention illustrating the degree of articulation about a horizontal axis extending through passageway 15;

FIG. 7B illustrates the humeral component as rotated from its position shown in FIG. 7A through approximately 120°, which approximates the range of motion in a healthy elbow joint;

FIG. 8 is an illustration of a healthy ulna illustrating the trochlear notch, which provides side-to-side support for the joint in a healthy, natural elbow; and

FIG. 9 is a side elevational view showing the relative alignment of the humerus, ulna and radius in a normal healthy joint, and illustrating the trochlear notch.

### DETAILED DESCRIPTION OF THE DRAWINGS

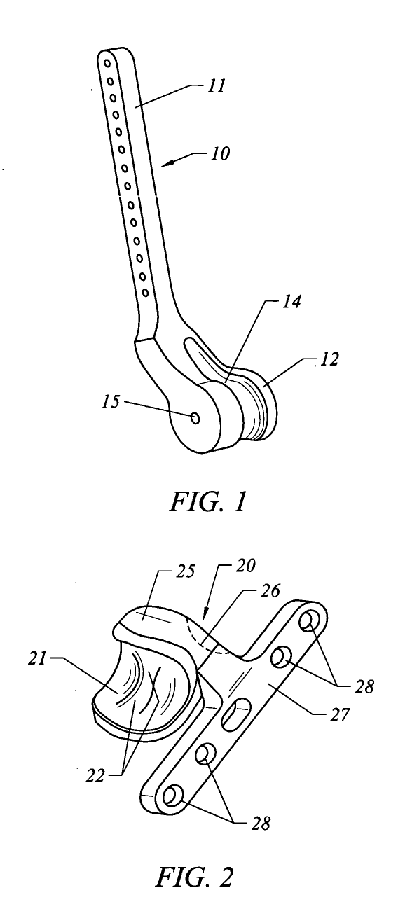

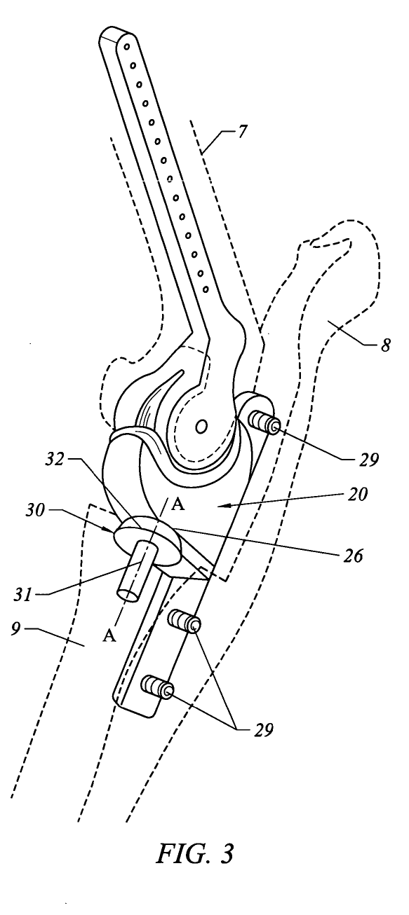

FIGS. 1-2 are perspective views illustrating two of the components of the present invention and their placement in the elbow is illustrated in FIG. 3.

FIG. 1 illustrates the humeral component shown generally as 10 having an elongated stem 11 which extends into the humerus and is cemented in place. The head 12 of the humeral component is generally cylindrical, similar to the head of the humerus bone, itself, and has a spiral groove 14 which extends around the head portion 12 and is described in greater detail below. A passageway 15 extends transversely through head portion 12. Passageway 15 is used only to position the humeral component 10 as the prosthesis is surgically implanted, and does not carry a pin.

The radial ulnar component shown generally as 20 in FIG. 2 is illustrated upside down relative to how it is shown in FIG. 3, for the purpose of illustrating the formed plastic seat 21 against which the head 12 of the humeral component sits and articulates. The plastic seat 21 has a ridge 22 extending vertically and is adapted to engage the spiral groove 14 in the humeral head 12. When the humeral component 10 is mated with the radial ulnar component 20, the joint is unconstrained, i.e., no pin or hinge holds the two components together. Rather the components are held together by ligaments as well as gravitational forces.

The radial ulnar component 20 has a body portion 25, preferably made of metal, to support the plastic seat 21. An anchor plate 27 is connected to body 25 and is provided with a plurality of passageways 28 which receive screws to connect the radial ulnar component 20 to the ulna bone as shown in FIG. 3.

As shown in FIG. 3, the humerus is shown in phantom as 7. The ulna is shown in phantom as 8 and the radius as 9. As shown in FIG. 3, the radial ulnar component 20 is anchored to the ulna 8 by a plurality of screws 29 which extend through passageways 28.

The third component of the present invention is the radial cap 30 which includes a generally cylindrical stem portion 31 and head 32. Stem portion 31 extends into the radius 9 after the diseased head of the radius has been cut off. The radial cap 30 has a domed head 32 which fits into a concave recess 26, shown in phantom in FIG. 2, formed in the distal end of the radial ulnar component. The radial cap 30 bears approximately 80% of the weight transferred by the dog's humerus downwardly, which is the same percentage carried by a healthy radius in a natural elbow joint. The ulna carries approximately 20% of the weight borne by the joint.

A significant aspect of the present invention is that the prosthesis is designed to allow the ulna 8 and radius 9 to rotate relative to each other about their longitudinal axes to allow pronation or supination of the animal's carpus, as discussed generally above. The radial stem 31 is implanted with its longitudinal axis lying on axis A-A, which is the longitudinal axis of rotation of radius 9.

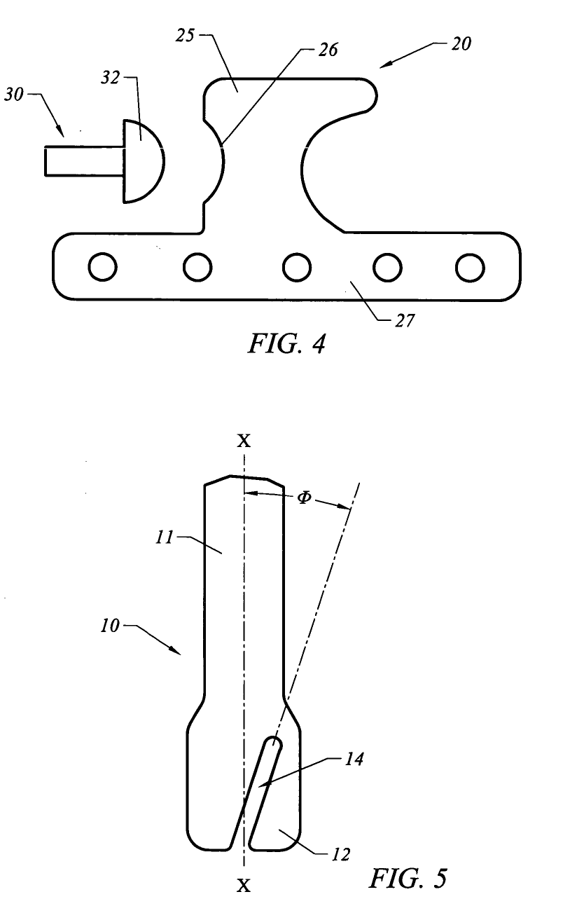

FIG. 4 illustrates how the concavity 26 formed in the body 25 of radial ulnar component 20 is designed to interact with the domed head 32 of radial cap 30.

FIG. 5 illustrates how spiral groove 14 formed in the head 12 of humeral component 10 is inclined at an angle φ relative to the longitudinal axis X-X of humeral component 10. The angle φ is approximately 13 degrees. By forming the spiral groove 14 at the angle 1, the humerus moves medially with respect to the radius 9 and ulna 8 as the joint extends while bearing weight, closely mimicking the motion of a natural elbow.

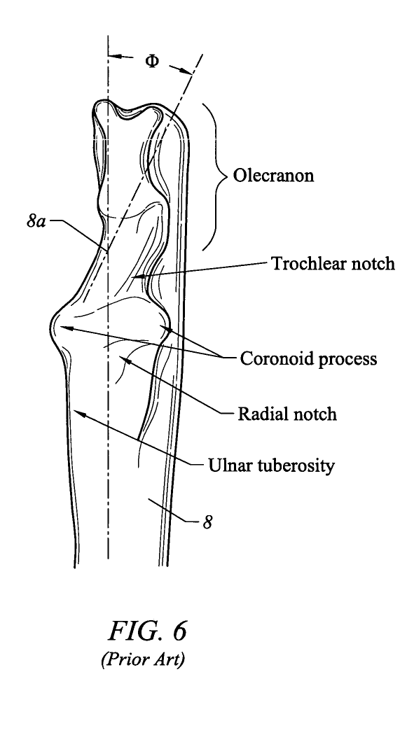

FIG. 6 is a cranial view of a natural, healthy left ulna, illustrating the inclination of the trochlear notch 8a at an angle φ relative to vertical axis Y-Y. This incline is utilized in the present invention to mimic the natural motion of the joint in weight-bearing extension. The present invention achieves the inclination φ by orienting ridge 22 (FIG. 2) vertically, and forming spiral groove 14 at an angle φ (FIG. 5). The natural joint has an inclined trochlear notch and an essentially vertical groove in the distal end of the humerus.

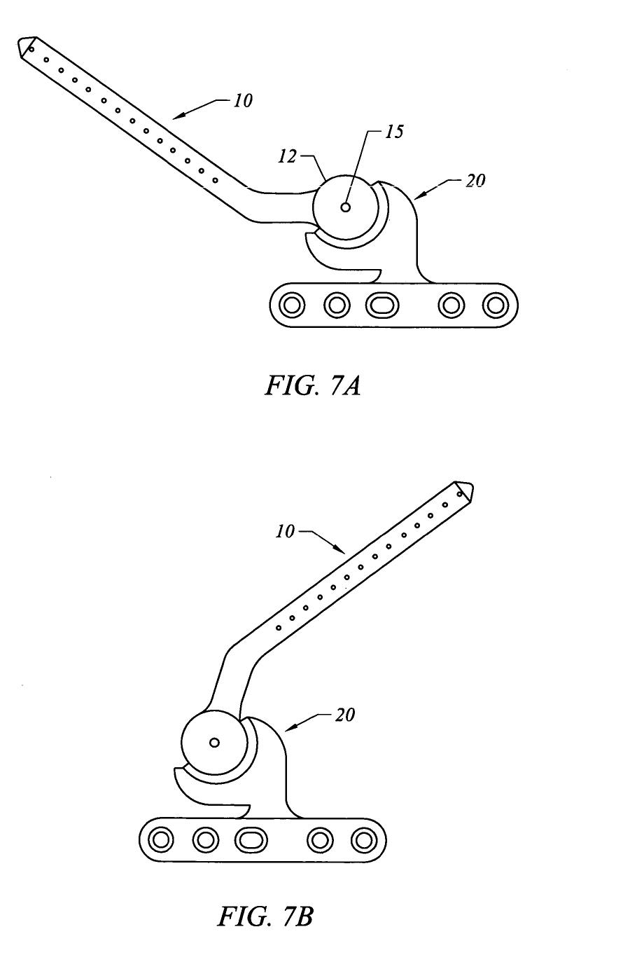

FIGS. 7A and 7B illustrate the degree of articulation about a horizontal axis extending through passageway 15, which is the axis of horizontal rotation of the joint of the humeral component 10 relative to the radial ulnar component 20. The total rotation allowed by the present invention is approximately 120 degrees which is equivalent to the natural elbow joint in a healthy dog.

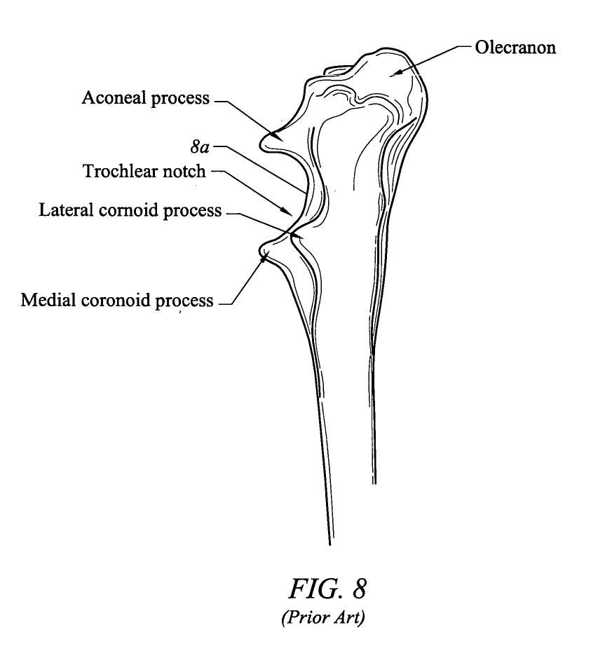

FIG. 8 illustrates a healthy ulna 8 and illustrates the trochlear notch 8a. In a healthy elbow joint, the head of the humerus bears against the trochlear notch 8a. As the ulna rotates, the relationship of the trochlear notch extends into the grooved head of the humerus and provides significant side-to-side or lateral support for the joint as it articulates.

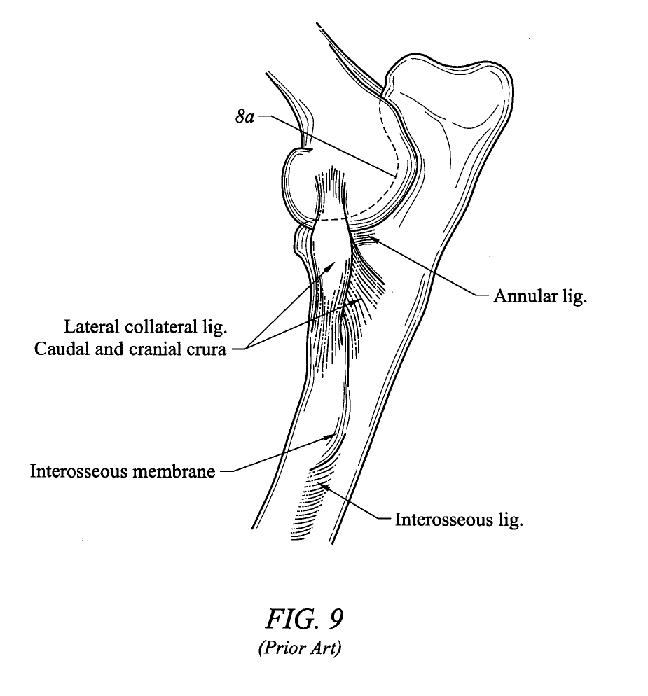

FIG. 9 shows the relative alignment of the humerus 7, ulna 8 and radius 9 in a normal healthy joint, as well as some of the ligaments. The trochlear notch 8a is shown in phantom, illustrating the side-to-side or lateral support provided in a natural joint. This lateral support is provided in the present invention by ridge 22 mimicking trochlear notch 8a and spiral groove 14 mimicking the olecranon fossa (or grooved end) of the natural humerus.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated.

What is claimed is:

1. A total elbow replacement prosthesis for dogs, wherein the ulna is left intact, wherein the distal end of the humerus is surgically removed, and the proximal end of the radius is surgically removed, comprising:
a humeral component having an elongated stem adapted to be carried by said humerus, having a generally cylindrical head carried by said stem, and a groove formed in said cylindrical head,
radial ulnar component having a body and a formed plastic seat against which the head of said humeral component sits and articulates,
a vertical ridge formed in said plastic seat adapted to engage said spiral groove to provide lateral support for said prosthesis, and
means for connecting said radial ulnar component to said radius and ulnar bones.

2. The apparatus of claim 1 wherein said means for connecting said radial ulnar component comprises:
a recess formed in said body,
an anchor plate adapted to be attached to said ulna, and
a radial cap means carried by the proximal end of the radius, said radial cap means having a domed surface adapted to engage said recess, whereby said prosthesis is unconstrained and said radius and ulna are free to rotate relative to each other about their longitudinal axes.

3. A total elbow replacement prosthesis for dogs, wherein the ulna is left intact, wherein the distal end of the humerus is surgically removed, and the proximal end of the radius is surgically removed, comprising:
a humeral component having an elongated stem adapted to be carried by said humerus, having a generally cylindrical head carried by said stem, and a spiral groove formed in said cylindrical head at an angle which is substantially the same as the angle of the trochlear notch in a healthy ulna,
a radial ulnar component having a body and a formed plastic seat against which the head of said humeral component sits and articulates,
a vertical ridge formed in said plastic seat adapted to engage said spiral groove to provide lateral support for said prosthesis and to mimic the natural motion of a healthy elbow, and
means for connecting said radial ulnar component to said radius and ulnar bones.

4. The apparatus of claim 1 wherein said means for connecting said radial ulnar component comprises:
a recess formed in said body,
an anchor plate adapted to be attached to said ulna, and
a radial cap means carried by the proximal end of the radius, said radial cap means having a domed surface adapted to engage said recess, whereby said prosthesis is unconstrained and said radius and ulna are free to rotate relative to each other about their longitudinal axes, to allow pronation and supination of the carpus.

5. The apparatus of claim 4 wherein said angle of said spiral groove is approximately 13°.
